(19) United States
(12) Patent Application Publication
(10) Pub. No.: US 2004/0045098 A1
(43) Pub. Date: Mar. 11, 2004

(54) Use of particular aminosilicones as a post-treatment of processes for coloring keratin fibers with direct dyes or with oxidation dyes

(76) Inventor: Pascale Lazzeri, Levallois Perret (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/290,159

(22) Filed: Nov. 8, 2002

(30) Foreign Application Priority Data
Nov. 8, 2001 (FR) 01 14481

(51) Int. Cl. A61K 7/13

(52) U.S. Cl. 8/405

(57) ABSTRACT

A composition for the post-treatment of a process for coloring, such as with at least one coloring agent chosen from direct dyes and oxidation dyes, human keratin fibers such as human hair, comprising at least one particular aminosilicone as well as a process for coloring, with, for example, at least one coloring agent chosen from direct dyes and oxidation dyes, human keratin fibers such as hair, comprising a post-treatment with a composition comprising at least one particular aminosilicone.

This disclosure relates to the use, as a post-treatment for a process for coloring human keratin fibers such as hair with at least one coloring agent chosen from direct dyes and oxidation dyes, of a composition comprising at least one particular aminosilicone.

This disclosure also relates to a process for coloring, with at least one coloring agent chosen from direct dyes and oxidation dyes, human keratin fibers such as hair, comprising a post-treatment with a composition comprising at least one particular aminosilicone.

Two main types of processes for coloring keratin fibers exist: direct dyeing, using, in the presence or absence of oxidizing agents, direct dyes and/or pigments which are colored molecules, giving the fibers a temporary color that may fade out after shampooing a few times, and “oxidation dyeing” using oxidation dye precursors and an oxidizing agent, which may give the fibers a more resistant color than that obtained with the previous type of dyeing.

The use of an oxidizing agent generally may result in a certain level of degradation of the keratin fiber.

There is currently a very marked trend toward increasing the frequency of shampooing, which may be reflected by a more substantial degradation of the dyeing results between two applications.

There is thus a need to improve the resistance of colorations with direct dyes or with oxidation dyes, for example, with respect to shampooing.

After extensive research, the inventors have discovered, entirely surprisingly and unexpectedly, that the use, as a post-treatment on human keratin fibers such as hair, of a composition comprising at least one particular aminosilicone, may allow this problem to be solved. This discovery forms at least a portion of the basis for at least one embodiment disclosed herein.

In addition, this post-treatment may improve the condition of the fiber, for example, in the case of a prior coloration in the presence of an oxidizing agent.

As used herein, the phrase “improvement in the condition of the fiber” means a reduction in the porosity or the alkaline solubility of the fiber and an improvement in at least one cosmetic property, for example, in the smoothness, softness and ease of disentangling and of styling.

This effect can be remanent, i.e., long-lasting.

The porosity is measured by fixing at 37° C. and at pH 10, for two minutes, 2-nitro-para-phenylenediamine at 0.25% in an ethanol/pH 10 buffer mixture (10/90 volume ratio).

The alkaline solubility corresponds to the loss of mass of a sample of 100 mg of keratin fibers under the action of decinormal sodium hydroxide for 30 minutes at 65° C.

A new embodiment relates to a composition for the post-treatment of a process for coloring, with at least one coloring agent chosen from oxidation dyes and direct dyes, human keratin fibers such as hair, comprising at least one aminosilicone comprising at least one aminoethylimino(C₄-C₈)alkyl group.

In one embodiment, the composition may improve the resistance to shampooing of said colorations and/or the condition of the fiber after coloration, for example, in the case of coloring with an oxidizing agent. This post-treatment may take place immediately after dyeing and optional rinsing, or after an interval, and may be performed once only or repeatedly between two colorations.

Another aspect of this disclosure relates to a coloring process that comprises applying to human keratin fibers, such as hair, a dye composition comprising at least one coloring agent chosen from oxidation dyes and direct dyes for a time that is sufficient to develop the color, and in following this application, after optionally rinsing, and after optionally drying, with the application of a composition comprising at least one aminosilicone comprising at least one aminoethylimino(C₄-C₈)alkyl group.

Aminosilicones

The at least one aminosilicone comprising at least one aminoethylimino(C₄-C₈)alkyl group has, for example, the following formula:

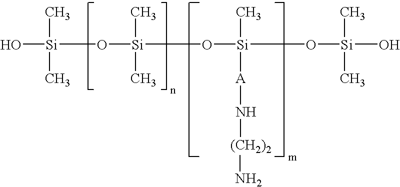

wherein:

A is chosen from linear and branched C₄-C₈ alkylene radicals, for example, C₄ alkylene radicals and

m and n are numbers such that the sum (n+m) can range, for example, from 1 to 2000 and further, for example, from 50 to 150, n may be a number ranging from 0 to 1999, for example, from 49 to 149, and m may be a number ranging from 1 to 2000, for example, from 1 to 10.

The term “alkylene radical” means divalent saturated hydrocarbon-based groups.

The viscosity of the at least one aminosilicone, for example, can be greater than 25 000 mm²/s at 25° C.

For example, this viscosity can range from 30 000 to 200 000 mm²/s at 25° C. and further, for example, from 30 000 to 150 000 mm²/s at 25° C.

The viscosity of the at least one aminosilicone is measured at 25° C. according to the standard “ASTM 445 Appendix C”.

The at least one aminosilicone has a weight-average molecular mass, for example, ranging from 2000 to 1 000 000 and further, for example, from 3500 to 200 000.

The weight-average molecular masses of the at least one aminosilicone is measured by Gel Permeation Chromatography (GPC) at room temperature, as polystyrene equivalents. The columns used are styragel μ columns. The eluent is THF and the flow rate is 1 ml/minute. 200 μl of a solution at 0.5% by weight of silicone in THF are injected. The detection is performed by refractometry and UV-metry.

When at least one aminosilicone is used, one embodiment involves using the at least one aminosilicone in the form of an oil-in-water emulsion. The oil-in-water emulsion may comprise at least one surfactant. The at least one surfactant may be of any nature, for example, cationic and/or nonionic.

The silicone particles in the emulsion may have a mean size ranging, for example, from 3 to 500 nanometers and further, for example, from 5 to 300 nanometers, even further, for example, from 10 to 275 nanometers and even further, for example, from 150 to 275 nanometers. Such particle sizes are measured with a laser granulometer.

An example of a silicone corresponding to this formulation is DC2-8299® from the company Dow Corning.

Another embodiment uses at least one aminosilicone in the post-treatment composition in an amount ranging from 0.01% to 20% by weight relative to the total weight of the composition. For example, this amount may range from 0.1% to 15% by weight and further, for example, from 0.5% to 10% by weight relative to the total weight of the composition.

The post-treatment composition may comprise any ingredient conventionally used in cosmetics, such as in the field of haircare. For example, it may comprise at least one additional surfactant and/or polymer. These surfactants and polymers may be chosen from nonionic, cationic, anionic and amphoteric surfactants and polymers. Among the additional polymers, aminosilicones other than those disclosed herein, may be used.

The post-treatment composition may have a pH ranging from 2 to 11, for example, from 4 to 9.

The post-treatment composition may be in various forms, such as lotions, gels, creams, shampoos, sticks, mousses and sprays. For some of these forms, it may be packaged in a pump-dispenser bottle or in an aerosol container. In the case of an aerosol, the composition may be combined with a propellant that may be, for example, an alkane or a mixture of alkane, dimethyl ether, nitrogen, nitrous oxide, carbon dioxide and haloalkanes, and also mixtures thereof.

In one new embodiment, the post-treatment composition may be in shampoo form.

When the post-treatment composition is in shampoo form, the composition comprises at least one surfactant, for example, an anionic surfactant. The post-treatment composition may also comprise a mixture of surfactants comprising at least one anionic surfactant and at least one other surfactant chosen from nonionic and amphoteric surfactants.

As mentioned above, the post-treatment composition may be applied immediately after coloration, or after an interval. The expression “after an interval” means an application performed a few hours, one day or several days, for example, from 1 to 60 days, after the coloration.

According to one new embodiment, several applications can be carried out between two colorations.

The number of applications between two colorations can range, for example, from 1 to 60 and further, for example, from 2 to 30.

The post-treatment composition may be used in rinse-out or leave-in mode, i.e., its application may or may not be followed by a rinsing operation.

In one new embodiment, the acting time of the post-treatment composition ranges from few seconds to 60 minutes, for example, from 30 seconds to 15 minutes.

The application temperature of the post-treatment composition may range from 10° C. to 70° C. For example, the application temperature may range from 10° to 60° C., such as at room temperature.

The nature and concentration of the dyes present in the dye composition is not critical. In the case of colorations with direct dyes (in the presence or absence of oxidizing agents), the dye compositions may comprise at least one dye chosen from neutral, acidic and cationic nitrobenzene direct dyes, neutral, acidic and cationic azo and methine direct dyes, neutral, acidic and cationic quinone direct dyes, for example, anthraquinone direct dyes, azine direct dyes, triarylmethane direct dyes, indoamine direct dyes, natural direct dyes and mixtures thereof.

In the case of colorations with oxidation dyes, the dye compositions may comprise at least one oxidation base.

The at least one oxidation base may be chosen from those conventionally used in oxidation dyeing, and among which mention may be made, for example, of ortho-phenylenediamines, para-phenylenediamines, double bases, ortho-aminophenols, para-aminophenols, heterocyclic bases, and their acid addition salts.

The oxidation dye compositions may also comprise at least one coupler.

Representatives of the at least one coupler can include, for example, meta-phenylenediamines, meta-aminophenols, meta-diphenols, mono- and polyhydroxylated naphthalene derivatives, sesamol and its derivatives, and heterocyclic compounds such as, for example, indole couplers, indoline couplers and pyridine couplers, and their acid addition salts.

The nature of the oxidizing agent used in the lightening direct dyeing (direct dyeing with an oxidizing agent) or in the oxidation dyeing is not critical.

The at least one oxidizing agent may be chosen, for example, from hydrogen peroxide, urea peroxide, alkali metal bromates, alkali metal ferricyanides, and persalts such as perborates and persulfates. At least one redox enzyme such as laccases, peroxidases and 2-electron oxidoreductases (such as uricase) may also be used as an oxidizing agent, where appropriate in the presence of the respective donor or cofactor thereof.

The examples that follow are intended to illustrate in a non-limiting way embodiments disclosed herein.

### EXAMPLES

The three post-treatment compositions A, B and C below were prepared.

(Expressed as Grams of Active Material (AM))

| Composition A | |
|---|---|
| Polydimethylsiloxane: DC2-8299 ® from the company | 2 |
| Dow Corning | |
| Demineralized water qs | 100 |
| Composition B | |
| Crosslinked homopolymer of ethyltrimethylammonium | 0.5 |
| methacrylate chloride as an inverse emulsion | |
| Hydroxypropyl corn distarch phosphate | 3 |
| Oxyethylenated (40 EO) hydrogenated castor oil | 0.5 |
| Polydimethylsiloxane: DC2-8299 ® from the company | 2.0 |
| Dow Corning | |
| Fragrance | 0.3 |
| Preserving agents | 0.3 |
| Demineralized water qs | 100 |
| Composition C | |
| Sodium lauryl ether sulfate comprising 2.2 mol of ethylene oxide | 7 |
| Cocoylbetaine | 2.5 |
| Glycol distearate | 1.5 |
| Polydimethylsiloxane: DC2-8299 ® from the company | 1.8 |
| Dow Corning | |
| Hydroxyethylcellulose quaternized with 2,3- | 0.4 |
| epoxypropyltrimethylammonium chloride, sold under the brand | |
| name Ucare Polymer JR-400 ® by Union Carbide | |
| Acrylic polymer as an emulsion sold under the brand name Aqua | 0.8 |
| SF1 ® by Noveon | |
| Preserving agents | qs |
| pH agents qs | pH 5 |
| Demineralized water qs | 100 |

Compositions A and B were applied for two minutes to locks of moderately bleached hair dyed with the commercial oxidation dye Majirouge®, shade 7.40.

After rinsing and drying, five standard shampoo washes were carried out on these locks.

For comparative purposes, locks dyed under the same conditions, and subjected to the same shampooing protocol, were not subjected to post-treatment with compositions A and B.

Results: the degradation of the color relative to that of a lock of unshampooed hair is less in the case of the post-treatments with compositions A and B than in the case where a post-treatment was not carried out.

Moreover, the condition of the hair fibers was satisfactory.

Composition C is an illustrative form of post-treatment in shampoo form.

It was used as a care shampoo on colorations with oxidation dyes or with direct dyes. The degradations of the initial shades were less than in the case where a standard care shampoo was used (without the aminosilicone of the invention).

The condition of the hair fibers was judged to be better.

What is claimed is:

1. A process for coloring human keratin fibers comprising
applying to said fibers a composition comprising at least one coloring agent chosen from direct dyes and oxidation dyes and
thereafter, applying to said colored fibers, a post-treatment composition comprising at least one aminosilicone comprising at least one aminoethylimino(C₄-C₈)alkyl group.

2. The process according to claim 1, wherein said human keratin fibers are hair.

3. The process according to claim 1, wherein the at least one aminosilicone comprising at least one aminoethylimino(C₄-C₈)alkyl group has the following formula: 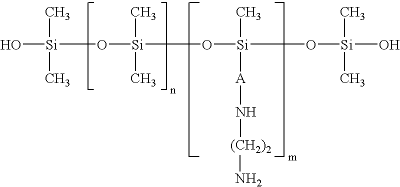
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000.

4. The process according to claim 3, wherein the sum (n+m) ranges from 50 to 150.

5. The process according to claim 3, wherein n is a number ranging from 49 to 149.

6. The process according to claim 3, wherein m is a number ranging from 1 to 10.

7. The process according to claim 3, wherein A is chosen from linear and branched C₄ alkylene radicals.

8. The process according to claim 3, wherein the viscosity of the at least one aminosilicone is greater than 25 000 mm²/s at 25° C.

9. The process according to claim 8, wherein the viscosity of the at least one aminosilicone ranges from 30 000 to 200 000 mm²/s at 25° C.

10. The process according to claim 9, wherein the viscosity of the at least one aminosilicone ranges from 30 000 to 150 000 mm²/s at 25° C.

11. The process according to claim 1, wherein the at least one aminosilicone has a weight-average molecular mass ranging from 2000 to 1 000 000.

12. The process according to claim 11, wherein the at least one aminosilicone has a weight-average molecular mass ranging from 3500 to 200 000.

13. The process according to claim 1, wherein the at least one aminosilicone is in the form of an oil-in-water emulsion comprising at least one surfactant.

14. The process according to claim 13, wherein the oil-in-water emulsion comprises at least one surfactant chosen from cationic and nonionic surfactants.

15. The process according to claim 13, wherein the particle size of said at least one aminosilicone ranges from 3 to 500 nanometers.

16. The process according to claim 15, wherein the particle size of said at least one aminosilicone ranges from 5 to 300 nanometers.

17. The process according to claim 16, wherein the particle size of said least one aminosilicone ranges from 10 to 275 nanometers.

18. The process according to claim 17, wherein the particle size of said least one aminosilicone ranges from 150 to 275 nanometers.

19. The process according to claim 1, wherein the at least one aminosilicone is present in an amount ranging from 0.01% to 20% by weight relative to the total weight of the composition.

20. The process according to claim 19, wherein the at least one aminosilicone is present in an amount ranging from 0.1% to 15% by weight relative to the total weight of the composition.

21. The process according to claim 20, wherein the at least one aminosilicone is present in an amount ranging from 0.5% to 10% by weight relative to the total weight of the composition.

22. The process according to claim 1, wherein the post-treatment composition is provided in a form chosen from lotions, gels, creams, shampoos, sticks, mousses and sprays.

23. The process according to claim 1, wherein the post-treatment composition is packaged in a pump-dispenser bottle or in an aerosol container.

24. The process according to claim 23, wherein the post-treatment composition is combined with at least one propellant chosen from alkanes, dimethyl ether, nitrogen, nitrous oxide, carbon dioxide and haloalkanes.

25. The process according to claim 1, wherein the post-treatment composition comprises at least one surfactant chosen from nonionic, cationic, anionic and amphoteric surfactants.

26. The process according to claim 25, wherein the post-treatment composition comprises a mixture of surfactants comprising at least one anionic surfactant and at least one other surfactant chosen from nonionic and amphoteric surfactants.

27. The process according to claim 1, wherein the post-treatment composition comprises at least one additional polymer other than said at least one aminosilicone comprising at least one aminoethylimino(C₄-C₈)alkyl group.

28. The process according to claim 27, wherein said at least one additional polymer is chosen from nonionic, cationic, anionic and amphoteric polymers.

29. The process according to claim 28, wherein said at least one additional polymer is an aminosilicone different than said at least one aminosilicone.

30. The process according to claim 1, wherein the pH of the post-treatment composition ranges from 2 to 11.

31. The process according to claim 30, wherein the pH of the post-treatment composition ranges from 4 to 9.

32. The process according to claim 1, wherein said post-treatment composition is applied between two colorations.

33. The process according to claim 32, wherein said post-treatment composition is applied from 1 to 60 times between two colorations.

34. The process according to claim 33, wherein said post-treatment composition is applied from 2 to 30 times between two colorations.

35. The process according to claim 3, wherein said human keratin fibers are hair.

36. The process according to claim 1, wherein the post-treatment composition is left to act for a time ranging from a few seconds to 60 minutes

37. The process according to claim 36, wherein the post-treatment composition is left to act for a time ranging from 30 seconds to 15 minutes.

38. A process for improving the resistance of coloration on human keratin fibers comprising, after coloring said fibers with at least one coloring agent chosen from direct dyes and oxidation dyes, applying to said fibers a post-treatment composition comprising at least one aminosilicone comprising at least one aminoethylimino(C₄-C₈)alkyl group.

39. A process for improving the condition of human keratin fibers after coloration, comprising, after coloring said fibers with at least one coloring agent chosen from direct dyes and oxidation dyes, applying to said fibers a post-treatment composition comprising at least one aminosilicone comprising at least one aminoethylimino(C₄-C₈)alkyl group.

40. A process according to claim 39, wherein said post-treatment composition is applied after applying to said fibers a dye comprising an oxidizing agent.

41. A process for coloring human keratin fibers comprising applying to said fibers a composition comprising at least one coloring agent chosen from direct dyes and oxidation dyes,
leaving said composition on said fibers for a time sufficient to develop the color,
optionally rinsing and optionally drying said fibers, and
applying to said fibers, either immediately after said color is developed or after an interval following development of said color, a post-treatment composition comprising at least one aminosilicone comprising at least one aminoethylimino(C₄-C₈)alkyl group.

42. A composition for post-treatment of human keratin fibers after coloration with at least one coloring agent chosen from oxidation dyes and direct dyes, said composition comprising at least one aminosilicone comprising at least one aminoethylimino(C₄-C₈)alkyl group, wherein said composition is effective for the post-treatment of human keratin fibers after a process for coloring with said at least one coloring agent.
